(19) United States
(12) Patent Application Publication
(10) Pub. No.: US 2021/0386897 A1
(43) Pub. Date: Dec. 16, 2021

(54) WATER-SOLUBLE EFFERVESCENT SOLID PERFUME

(71) Applicant: MASSCON CO., LTD., Gyeonggi-do (KR)

(72) Inventors: Yongeui LEE, Gyeonggi-do (KR); Min Young CHEONG, Gyeonggi-do (KR); Chang Bin LEE, Busan (KR)

(21) Appl. No.: 17/290,919

(22) PCT Filed: Nov. 17, 2019
(86) PCT No.: PCT/KR2019/015014

§ 371 Date: May 3, 2021

(30) Foreign Application Priority Data
Nov. 8, 2018 (KR) 10-2018-0136338

(51) Int. Cl. A61L 9/012; A61L 9/014; A61L 9/04; A61L 2/23

(52) U.S. Cl. CPC A61L 9/012; A61L 9/014; A61L 9/046; A61L 2209/22; A61L 2202/17; A61L 2209/21; A61L 2/23

(57) ABSTRACT

The present invention relates to a water-soluble effervescent solid perfume comprising a high content of fragrance which does not include organic solvents such as surfactants, preservatives, and alcohols. In addition, additional components may be carried for cleaning and sterilizing effects, and the effects of fragrance, cleaning, sterilization, and the like may be increased by impartation of an effervescent property. Furthermore, there is no generation of residual by-products after effervescent, and may be processed into a solid formulation, thereby improving stability and improving portability and convenience of use.

### TECHNICAL FIELD

The present invention relates to a water-soluble effervescent solid perfume comprising a high content of a fragrant material that does not include surfactants, preservatives, and organic solvents such as alcohols, and provides immediate and rich diffusion of scent.

In addition, it relates to a water-soluble effervescent solid perfume that can impart cleaning and antibacterial/sterilizing effects and has stability even in a high humidity environment.

In addition, the present invention relates to a method for producing a water-soluble effervescent solid perfume as described above.

### BACKGROUND ART

Fragrances can change emotions of animals including humans, and has been used for a long time in aromatherapy to improve mental and physical health with a feeling of relief, pleasure, tension, arousal, meditation, etc. It is also essentially used to cover up or reduce unpleasant odors.

Conventionally, products such as liquid oil, scented candles, and diffusers are used, and for immediate diffusion of scent, fragrant materials are mixed with water, alcohol, etc., and a spray method is used.

In recent years, people's daily life is rapidly changing to mobile-based, and portability of such fragrance products that can be easily and conveniently used regardless of the place and time is essential. Conventionally, as a product that can be easily carried and easily used, a spray type product is used. The spray type product is the most widely used product for relaxation or for reducing unpleasant odors. Moreover, recently, a product that can cover unpleasant odors by spraying directly into the toilet bowl to form an oil film on the water in the toilet bowl are being released.

In order to manufacture such a spray type product, the use of synthetic additives such as surfactants and preservatives is indispensable because it is necessary to mix fragrance oils with water or organic solvents such as alcohols. The spray type product has problems such as flowing the contents or condensation of sprayed fine droplets on the floor or wall. In particular, since the sprayed fine droplets are diffused into the air, residual droplets can be absorbed into the body through the nose and mouth, and at this time, additives such as surfactants, preservatives, alcohols, etc., as well as fragrances can be absorbed together, which is harmful to humans and animals.

Therefore, it is preferable to diffuse only the fragrance component into the air, and a solid perfume that does not contain organic solvents such as alcohol or synthetic additives such as surfactants and preservatives is a good way to solve this problem.

However, conventional solid perfumes are mainly commercialized with impregnating a fragrance oil into a solid such as gypsum, clay, ceramic, etc. The problem is that the loading capacity of the fragrance oil is extremely low. In addition, in the case of these products, not only the immediate diffusion of scent is very poor due to the absence of alcohol, which is a scent diffusion aid, but also the richness and persistence of scent diffusion are very limited. Therefore, there are problems with the function as a fragrance product, that is, immediate diffusion of scent, the richness and persistence of scent diffusion.

Therefore, in the manufacture of a safe solid perfume that maximize the portability of the perfume and does not have the risk of inhalation of synthetic additives, which are harmful factors other than fragrance components, there is a need to develop a solid perfume capable of loading a high content of a fragrance oil with immediate diffusion of scent and rich and persistent scent diffusing capability.

### DETAILED DESCRIPTION OF THE INVENTION

### Technical Problem

It is an object of the present invention to provide a water-soluble effervescent solid perfume that supports a high content of a fragrance oil without synthetic additives such as surfactants, preservatives, alcohols, etc. in addition to the fragrance component to maximize the portability of the fragrance and increase the safety to the human body, and maximizes immediate diffusion of scent when used, and a method for producing the same.

### Solution to Problem

In order to solve the above problems, in the present invention, a high content of a fragrance oil is loaded into porous particles having nanopores, thereby providing richness and persistence of scent diffusion, and through bubbles generated during effervescent, fragrance components are dispersed in the aqueous system and released onto the surface of water, thereby providing immediate scent diffusion and diffusion of only fragrance components. In addition, the immediate scent diffusion and diffusion of only fragrance components are maximized by instantaneously increasing the temperature around the bubbles generated during effervescent. In addition, it is intended to additionally provide improved cleaning, antibacterial, and sterilizing effects by using such effervescent performance and to improve the moisture susceptibility of the effervescent component.

To this end, the present invention provides a water-soluble effervescent solid perfume comprising:

porous particles having a plurality of pores;

a fragrant material contained in the pores of the porous particles;

a binding agent for binding the porous particles containing the fragrant material to each other; and

an effervescent agent for dispersing the fragrant material in an aqueous system,

wherein a weight ratio of the porous particles to the binding agent is 1:1 to 30.

According to one embodiment, the effervescent agent may comprise an organic acid or an inorganic acid; and a carbonate salt, and specifically a weight ratio of the organic acid or inorganic acid:the carbonate salt may be 1:0.2 to 8.

According to one embodiment, the solid perfume may comprise, based on the total weight of the porous particles and the binding agent and the effervescent agent,

1 to 30% by weight of the porous particles;

25 to 65% by weight of the binding agent; and

25 to 65% by weight of the effervescent agent.

According to one embodiment, the porous particle may comprise at least one selected from the group consisting of silica, zeolite, activated carbon and acrylic resin particles.

According to one embodiment, the binding agent may comprise at least one selected from the group consisting of hydroxypropyl methyl cellulose, microcrystalline cellulose, starch and polyvinyl pyrrolidone.

According to one embodiment, an average particle size of the porous particle may be 1 to 100 μm.

According to one embodiment, an average pore diameter of the porous particle may be 1 to 100 nm.

According to one embodiment, the load rate of the fragrant material may be 30 to 500% as calculated according to Equation 1.

Load rate (%)=(a maximum weight of loaded fragrant material (g)/a total weight of solid (g))×100 [Equation 1]

In the above equation, the total weight of solid refers to the total sum of the weight of the porous particles, the weight of the binding agent, and the weight of the effervescent agent.

According to one embodiment, the structure formed by the porous particles and the binding agent may have voids and the void may have an average diameter 0.1 to 100 μm.

According to one embodiment, at least one selected from the group consisting of a cleaning agent, a sterilizer, and a deodorant may be further contained in the pores of the porous particles.

According to one embodiment, the perfume may further comprise magnesium sulfate.

According to another aspect of the present invention, there is provided a method for manufacturing a water-soluble effervescent solid perfume, comprising mixing 3 to 100 parts by weight of porous particles, 30 to 100 parts by weight of a fragrant material, and 30 to 300 parts by weight of an effervescent agent based on 100 parts by weight of the binding agent to produce a mixture.

According to one embodiment, the method may further comprise preparing the mixture into the form of a tablet or capsule.

According to one embodiment, the method may further comprise mixing 1 to 30% by weight of magnesium sulfate based on the total weight of the porous particles and the binding agent and the effervescent agent.

Other specifics of the embodiments of the present invention are included in the detailed description below.

### Effect of the Invention

According to the water-soluble effervescent solid perfume of the present invention and its manufacturing method, it is possible to provide a solid perfume comprising a high content of a fragrance oil without synthetic additives such as surfactants, preservatives, alcohols, etc. Therefore, it is possible to manufacture an eco-friendly water-soluble effervescent solid perfume that has richness of scent diffusion, allows immediate dissipation of fragrance, is highly convenient to use, is harmless to humans, and does not generate waste after use.

In addition, it is possible to additionally provide improved cleaning, antibacterial, and sterilizing effects by using such effervescent performance

### BEST MODE FOR CARRYING OUT THE INVENTION

Since various modifications and variations can be made in the present invention, particular embodiments are illustrated in the drawings and will be described in detail in the detailed description. It should be understood, however, that the invention is not intended to be limited to the particular embodiments, but includes all modifications, equivalents, and alternatives falling within the spirit and scope of the invention. In the following description of the present invention, detailed description of known functions will be omitted if it is determined that it may obscure the gist of the present invention.

Fragrances can change emotions of animals including humans, and has been used for a long time in aromatherapy to improve mental and physical health with a feeling of relief, pleasure, tension, arousal, meditation, etc. It is also essentially used to cover up or reduce unpleasant odors.

In recent years, fragrance products are also essential for portability that can be used easily regardless of the place and time, and a typical product that is portable and easy to use is in the form of a spray. In particular, recently, a product that can cover unpleasant odors by spraying directly into the toilet bowl to form an oil film on the water in the toilet bowl are being released. These spray products essentially contain organic solvents such as alcohol to mix fragrant materials with water, and synthetic additives such as surfactants and preservatives. These spray type products may have a harmful effect on humans or animals, as the sprayed droplets may get on the floor or wall or be inhaled into the human body.

Therefore, it is preferable to diffuse only the fragrance component into the air, and a solid perfume composition that does not contain synthetic additives such as surfactants and preservatives is required. Conventional solid perfume such as gypsum, clay, ceramic, etc. have extremely low loading capacity of fragrant materials. In addition, it has very poor immediate diffusion of scent low due to the absence of alcohol that is a scent diffusion aid, and the carrier is not decomposed after use and thus must be disposed of separately.

Therefore, there is a need for an eco-friendly composition of a water-soluble effervescent solid perfume that maximizes portability, does not have the risk of inhalation of synthetic additives, which are harmful factors other than fragrance components, comprises a high content of a fragrant material, has excellent performance of immediate diffusion of scent and does not generate waste after use in an aqueous system.

Hereinafter, the water-soluble effervescent solid perfume and the method of manufacturing the same according to embodiments of the present invention will be described in more detail.

As used herein, the term “binding agent” may be described interchangeably with “matrix”, “anchor” or “binder” and may refer to any material, for example having a fibrous structure, which can exist in inter-particle spaces to form a void.

As used herein, the term “fragrant material” may be used interchangeably with “aroma material” and may include, for example, an oil, a hydrophilic solution, or a combination thereof.

Unless otherwise specified in the disclosure, the expression “to” as used with numerical values means an expression including the corresponding numerical value. Specifically, for example, the expression “1 to 2” means not only including 1 and 2, but also including all numerical values between 1 and 2.

The present invention provides a water-soluble effervescent solid perfume comprising porous particles having a plurality of pores, a fragrant material contained in the pores of the porous particles, a binding agent for binding the porous particles containing the fragrant material to each other, and an effervescent agent for dispersing the fragrant material.

The effervescent agent includes a water-soluble effervescent agent that generates bubbles through foaming to disperse the fragrance component in an aqueous system and quickly discharges it onto the water surface, thereby enabling immediate diffusion of scent.

According to one embodiment, the porous particles may include at least one selected from the group consisting of silica, zeolite, activated carbon and acrylic resin particles. As the porous particle, it is preferable to use a material that is harmless to the human body and the environment, and may further include an additional carrier. Porous particles have a very large surface area by the formation of pores and form a three-dimensional skeleton structure, so that it is possible to support a high content of a fragrant material therein. In addition, in the case of making a tablet by mixing the fragrant material with the effervescent agent without porous particles, the fragrant material is filled in the space inside the tablet, thereby interfering with the reaction between water and the effervescent agent and deteriorating the effervescent characteristics and thus making difficult to immediately diffuse scent by foaming. The porous particles support the fragrance component as much as possible, so that the reaction between water and the effervescent agent occurs actively, which serves to maximize foaming properties. In this way, in order to comprise a high content of the fragrant material and enhance immediate effervescent characteristics and scent diffusing characteristics, the particles may have, an average pore diameter of, for example, 1 to 100 nm, for example, 10 to 100 nm, for example, 20 to 50 nm, and an average particle size (diameter) of 1 to 100 μm, for example, 1 to 10 μm. In addition, in order to improve the load rate, the porosity of the porous particles may be, for example, 70% or more, for example, 80% or more, for example, 90% or more.

According to one embodiment, the binding agent may include at least one selected from the group consisting of hydroxypropyl methyl cellulose (HPMC), microcrystalline cellulose (MCC), starch and polyvinyl pyrrolidone.

The binding agent may have, for example, a fibrous structure, and plays a role of stably binding porous particles to each other and is characterized by a lot of voids formed between structures entangled with particles. The average void diameter of the structure thus formed may be, for example, 0.1 to 100 μm, for example, 0.5 to 50 μm, for example, 1 to 20 μm. With such a void size, it can serve as a channel for fixing porous particles and for supporting and releasing the fragrant material. In addition, by including the binding agent, the porous particles do not have a flying phenomenon even after the fragrant material is completely discharged, so that the product can be safely used and the composition can be freely molded.

According to one embodiment, the fragrant material may include, for example, aroma essential oils or fragrance oils, and may include, for example, an extract extracted with a water-soluble solvent or a lipid-soluble solvent. Specific examples of essential oils and fragrance oils include oils obtained from one or more selected from the group consisting of lavender, grapefruit, geranium, cinnamon leaves, tea tree, cedarwood, orange, eucalyptus, bergamot, lemon, lime, mandarin, myrrh, neroli, niaouli, peppermint, pine, rosemary, chamomile, ylang-ylang, neem, frankincence, benzoin, helichrysum, phytoncide, rosewood, sandalwood, and the like. In addition, the fragrant material may be diluted with a carrier oil or a solvent. The carrier oil includes oils obtained from one or more selected from the group consisting of grapeseed, evening primrose, rosehip, macadamia nuts, borage, safflower, sesame, St. John's wort oil, sweet almond, avocado, apricot kernel, olive, wheatgerm, calendula, carrot, coconut, hazelnut, jojoba, basil and almond. In addition, the fragrant material may further comprise a solvent and an additive, the solvent may include ethanol, water, glycerin, silicone oil and the like, and the additive may include pigments. In the present invention, the fragrant material is not limited to the main fragrance substances as described above and any conventional vegetable oil may be used without limitation. Other solvents and additives generally used for combining fragrant materials are included without particular limitation.

According to one embodiment, the effervescent agent may include a carbonate salt together with an organic acid or an inorganic acid. The weight ratio of the organic or inorganic acid to the carbonate salt affects effervescent properties and molding stability. For example, the weight ratio of the organic acid or inorganic acid:carbonate salt may be 1:0.2 to 8, for example 1:0.3 to 5, for example 1:0.3 to 3. Examples of the organic acid include citric acid, tartaric acid, malic acid, malonic acid, pyridone carboxylic acid, succinic acid, fumaric acid, adipic acid, glutaric acid, and the like, and for example citric acid. The carbonate salt includes, for example, sodium hydrogen carbonate, sodium carbonate, sesqui-sodium carbonate, potassium carbonate, potassium hydrogen carbonate, ammonium carbonate, magnesium carbonate, and the like, and for example sodium hydrogen carbonate (NaHCO₃).

According to one embodiment, the above-described effervescent agent is added to enhance the immediate diffusion of scent due to foaming, and also when absorbing water, it emits heat to further accelerate foaming, thereby helping more immediate diffusion of scent. An effervescent agent such as magnesium sulfate may be included in the water-soluble solid perfume. General effervescent agents with cleaning and anti-bacterial/sterilizing effects tend to deteriorate storage stability in a high humidity environment. However, when magnesium sulfate with excellent moisture-retaining ability is added, magnesium sulfate absorbs moisture in the air, thereby minimizing the occurrence of an effervescent reaction during storage. As a result, storage stability in a high-humidity environment can also be improved simultaneously.

According to another embodiment of the present invention, the composition may comprise 1 to 30% by weight, for example 3 to 20% by weight of the porous particles; 25 to 65% by weight of the binding agent; 25 to 65% by weight of the effervescent agent, based on the total weight of the porous particles and the binding agent and the effervescent agent.

Since the water-soluble effervescent solid perfume must be able to stably contain a high content of the fragrant material in order to immediately emit a desired fragrance, the weight ratio of the porous particles and the binding agent may be set to 1:1 to 30, for example 1:2 to 10. The weight ratio of the porous particles and the binding agent may affect the load rate and molding stability. Specifically, when the weight ratio of the binding agent relative to the porous particles is too high, the molding stability is deteriorated, and when the weight ratio of the binding agent relative to the porous particles is too low, the content ratio of the porous particles, which are the main material to which the fragrant material is loaded, may be lowered, thereby lowering the load rate.

According to another aspect of the present invention, there is provided a method for manufacturing a water-soluble effervescent solid perfume, comprising mixing 3 to 100 parts by weight, for example 10 to 50 parts by weight of porous particles, 30 to 300 parts by weight, for example 70 to 250 parts by weight of a fragrant material, and 30 to 300 parts by weight, for example 35 to 250 parts by weight of an effervescent agent based on 100 parts by weight of the binding agent to produce a mixture.

According to one embodiment, the water-soluble effervescent solid perfume of the present invention may have a load rate of the fragrant material of 30% or more, or 40% or more, and 500% or less, or 100% or less as calculated according to Equation 1.

Load rate (%)=(a maximum weight of loaded fragrant material (g)/a total weight of solid (g))×100 [Equation 1]

In the above equation, the total weight of solid refers to the total sum of the weight of the porous particles, the weight of the binding agent, and the weight of the effervescent agent.

According to one embodiment, in order to impart effects such as cleaning and anti-bacterial/sterilization effects, at least one selected from the group consisting of a cleaning agent, a sterilizer, and a deodorant may be further included in the pores of the porous particles of the composition. In particular, in order to impart the cleaning and anti-bacterial/sterilization effects and the immediate dissipation of fragrance in the water system, a cleaning agent and a sterilizer which have effervescent properties may be used together.

The cleaning agent and the sterilizer are not particularly limited as long as they are commonly used, and are preferably eco-friendly and human-friendly, and they may be included in a range not to impair the effects of the present invention.

According to another embodiment of the present invention, in addition to imparting an immediate dissipation of fragrance by the effervescent properties of the cleaning agent and sterilizer, it is possible to achieve more immediate diffusion of scent by additionally adding magnesium sulfate, etc. that emits heat when reacting with water to maximize the effervescent reaction. Moreover, the addition of magnesium sulfate, etc., which has excellent moisture-containing function, can provide storage stability in a high-humidity environment. The content of magnesium sulfate may be 90 parts by weight or less, for example 70 parts by weight or less, for example 50 parts by weight or less and 5 parts by weight or more, and 10 parts by weight or more based on 100 parts by weight of the binding agent. In addition, with respect to the total weight of the porous particles and the binding agent and the effervescent agent, magnesium sulfate is 1 to 30% by weight, for example, 25% by weight or less, or 20% by weight or less, and 2% by weight or more, or 3% by weight or more, or 4% by weight or more. If the content of magnesium sulfate is too high, the load rate of the fragrant material may decrease.

According to one embodiment, the water-soluble effervescent solid perfume according to the present invention may be provided in the form of a tablet or capsule, thereby improving convenience of use and portability.

Hereinafter, embodiments of the present invention will be described in detail so that those skilled in the art can easily carry out the present invention. The present invention may, however, be embodied in many different forms and should not be construed as limited to the embodiments set forth herein.

### EXAMPLES AND COMPARATIVE EXAMPLES

A water-soluble effervescent solid perfume was prepared according to the composition shown in Table 1. The content is expressed in units of gram.

As the porous particles, porous silica (average particle diameter: 10 μm or less) was used.

The porous particles, the effervescent agent and the binding agent were uniformly mixed. After mixing, 1 g of the mixture was put into a mold capable of tableting, and a certain pressure was applied to prepare a tablet.

The molding stability of the tablet was evaluated according to criteria of Table 2, and the foamability was evaluated according to criteria of Table 3 by putting the tablet into a container containing 1 L of purified water, and the results are shown in Table 1.

TABLE 1

| | | Effervescent agent | | | | | | | |
|---|---|---|---|---|---|---|---|---|---|
| | Porous | | Sodium | | | Content of | Content of | | |
| | micro- | Citric | hydrogen | Binding agent | | Effervescent | binding | Molding | Effervescent |
| Ex. | particles | acid | carbonate | Type | Content | agent(%) | agent(%) | stability | property |
| Comp. | 2 | 8 | 8 | MCC | 4 | 72.7 | 18.2 | X | ⊚ |
| Ex. 1 | | | | | | | | | |
| Comp. | 2 | 2 | 2 | MCC | 16 | 18.2 | 72.7 | ⊚ | X |
| Ex. 2 | | | | | | | | | |
| Comp. | 2 | 1 | 9 | MCC | 10 | 45.5 | 45.5 | ◯ | X |
| Ex. 3 | | | | | | | | | |
| Comp. | 2 | 9 | 1 | MCC | 10 | 45.5 | 45.5 | ⊚ | X |
| Ex. 4 | | | | | | | | | |
| Ex. 1 | 2 | 5 | 5 | MCC | 10 | 45.5 | 45.5 | ⊚ | ⊚ |
| Ex. 2 | 2 | 7 | 7 | MCC | 6 | 27.3 | 63.6 | ◯ | ⊚ |
| Ex. 3 | 2 | 3 | 3 | MCC | 14 | 63.6 | 27.3 | ⊚ | ◯ |
| Ex. 4 | 2 | 3 | 7 | MCC | 10 | 45.5 | 45.5 | ⊚ | ⊚ |
| Ex. 5 | 2 | 7 | 3 | MCC | 10 | 45.5 | 45.5 | ⊚ | ⊚ |

TABLE 2
Criteria for evaluating molding stability of tablet

| | |
|---|---|
| ⊚ | Very good |
| ◯ | Good |
| Δ | Powder comes out front the surface |
| X | Breaking into small particles |

TABLE 3
Criteria for evaluating molding stability of tablet

| | |
|---|---|
| ⊚ | Effervescent completed within 10 seconds, no residue after |
| | completion of effervescent |
| ◯ | Effervescent completed within 30 seconds, no residue after |
| | completion of effervescent |
| Δ | Effervescent for at least 1 minute, no residue after completion |
| | of effervescent |
| X | Effervescent for at least 1 minute, existence of residue after |
| | completion of effervescent |

Table 1 shows Comparative Examples and Examples for confirming the influence on the molding stability and effervescent property depending on the compositions of the porous rnicroparticles, effervescent agent and binding agent, without the loaded fragrant material. As shown in the results of Table 1, in the case that a certain amount of porous microparticles is included, that is, in the case that 1 to 30% by weight of porous particles are included with respect to the total weight of the porous particles and the binding agent and the effervescent agent, when the content of the binding agent is small, moldability is deteriorated, and when the content of the binding agent is excessive, the total content of the effervescent agent is lowered and thus the foamability is lowered. In particular, in Comparative Example 3 in which a content ratio of the organic acid or inorganic acid to the carbonate salt, both of which constitute the effervescent agent, is 0.1 and in Comparative Example 4 in which the content ratio is 9, it is found that the molding stability and foamability are greatly deteriorated. It can be seen that both molding stability and effervescent properties are excellent when the ratio is in the range of 0.2 to 8 as in the Examples.

According to the composition shown in Table 4, a water-soluble foamed solid perfume loaded with a fragrant material was prepared. After preparing a water-soluble effervescent solid perfume tablet in the same manner as in Example 1, the prepared tablet was put into the container containing lavender essential oil for 1 day to prepare an effervescent solid perfume loaded with a fragrant material. The tablet was put into a perfume container for 1 day and then taken out to analyze the loading amount.

TABLE 4

| | | | | | | Fragrant material | |
|---|---|---|---|---|---|---|---|
| | | Effervescent agent | | | | | Loading |
| | Porous | | Sodium | | | | amount |
| | micro- | Citric | hydrogen | Binding agent | | | (g/Tablet |
| Ex. | particles | acid | carbonate | Type | Content | Type | 1 g, Max) |
| Comp. | X | 5 | 5 | MCC | 10 | Lavender | 0.20 |
| Ex. 5 | | | | | | | |
| Comp. | 0.5 | 5 | 5 | MCC | 10 | Lavender | 0.25 |
| Ex. 6 | | | | | | | |
| Comp. | 8 | 5 | 5 | MCC | 10 | Lavender | — |
| Ex. 7 | | | | | | | |
| Ex. 6 | 2 | 5 | 5 | MCC | 10 | Lavender | 0.45 |
| Ex. 7 | 1 | 5 | 5 | MCC | 10 | Lavender | 0.37 |
| Ex. 8 | 5 | 5 | 5 | MCC | 10 | Lavender | 0.90 |
| Ex. 9 | 2 | 5 | 5 | HPMC | 10 | Lavender | 0.43 |

In addition, the molding stability and effervescent property were evaluated according to criteria in Tables 2 and 3, the load rate was calculated according to Equation 1, and the scent stability during storing tablet was evaluated according to criteria in Table 5, and the results are shown in Table 6. In addition to the scent stability of the tablet itself, the effervescent properties and the scent strength after the tablet was foamed in water were evaluated, and the results are shown in Table 6. Specifically, the scent stability and the post-effervescent scent intensity were evaluated by sensory evaluation by odor-trained evaluators. For the scent stability, the initial intensity of scent of the tablet comprising the fragrant material was evaluated and the scent intensity of the tablet after putting into a 70 ml vial and storing at 25° C. for 5 days with the lid open was evaluated. For the post-effervescent scent intensity, one tablet (1.5 g) comprising the fragrant material was put into a beaker containing 500 nil of water, and when the effervescent was completed the scent intensity was evaluated according to the criteria in Table 5.

Load rate (%)=(a maximum weight of loaded fragrant material (g)/a total weight of solid (g))×100 [Equation 1]

In the above equation, the total weight of solid refers to the total sum of the weight of the porous particles, the weight of the binding agent and the weight of the effervescent agent.

TABLE 5
Criteria of scent intensity

| Grade | |
|---|---|
| 0 | Odorless |
| 1 | Smell that can be felt slightly (minimum detectable |
| | concentration) |
| 2 | Weak smell to know what it is (minimum cognitive |
| | concentration) |
| 3 | Easily recognizable smell |
| 4 | Irritating smell |
| 5 | Strong smell |

TABLE 6

| | | | Post- | | | |
|---|---|---|---|---|---|---|
| | | | effervescent | Load | Scent stability (change | |
| | Molding | Effervescent | scent | rate | in scent intensity) | |
| Ex. | stability | property | intensity | (%) | Initial | After 5 days |
| Comp. | ⊚ | X | 2 | 20 | 0 | 1 |
| Ex. 5 | | | | | | |
| Comp. | ⊚ | X | 3 | 25 | 3 | 2 |
| Ex. 6 | | | | | | |
| Comp. | X | ◯ | — | — | — | — |
| Ex. 7 | | | | | | |
| Ex. 6 | ⊚ | ◯ | 4 | 45 | 4 | 4 |
| Ex. 7 | ⊚ | ◯ | 4 | 37 | 4 | 4 |
| Ex. 8 | ◯ | ⊚ | 5 | 90 | 5 | 5 |
| Ex. 9 | ◯ | ◯ | 4 | 43 | 4 | 4 |

As shown in Table 6, it can be seen that the load rate and the scent stability as well as the effervescent property of the water-soluble effervescent solid perfume according to Examples were improved compared to the those of Comparative Examples.

From the above results, it is found that when the porous microparticles capable of loading fragrant materials are not included or the content thereof is small as in Comparative Examples 5 and 6, the load rate of the fragrant material and the stability were low and the foamability was also lowered, resulting in a decrease in immediate diffusion of scent. On the other hand, it can be seen that the perfumes according to Examples 6 to 9 contain a high content of the fragrant material and have the improved foamability, compared to those of Comparative Examples 5 and 6, so that immediate dissipation of fragrance is possible. In the case of Comparative Examples 5 and 6, it can be seen that the fragrant material is filled in the space inside the solid perfume, so that the penetration of water into the solid perfume is reduced, and this decrease in the water penetration rate leads to a decrease in the effervescent rate and also the decrease in the effervescent rate leads to a decrease in scent diffusion rate. On the other hand, in the case of including more than a certain amount of porous microparticles as in Examples 6 to 9, a high content of the fragrant material is mostly loaded on the porous microparticles, and a space through which water can penetrate can be secured in the binding agent and the effervescent agent. Therefore, it can be seen that instant effervescent is possible by the reaction between water and the effervescent agent, thereby enhancing the diffusion of scent.

However, as in Comparative Example 7, if the content of the porous particles in the composition is too high, tablet molding stability is deteriorated, and thus it is difficult to manufacture into a form suitable for portable use.

According to the composition shown in Table 7, a water-soluble effervescent solid perfume loaded with a fragrant material was prepared. A tablet of the water-soluble effervescent solid perfume was prepared in the same manner as in Example 1, and then the prepared tablet was added to a container containing lavender essential oil and left to stand for 1 day to prepare an effervescent solid perfume loaded with a fragrant material. The tablet which had been put in the perfume container for 1 day, was taken out and the load rate was measured. Molding stability and effervescent properties of the tablet were evaluated according to the criteria in Tables 2 and 3, respectively, and the post-effervescent scent intensity after manufacture was evaluated according to the criteria in Table 5. In order to evaluate the storage stability in a high humidity environment, the tablet loaded with a fragrant material was stored for 12 hours in a constant temperature and humidity chamber set at 40° C. and RH 80%, and then the post-effervescent scent intensity was evaluated according to the criteria in Table 5.

TABLE 7

| | | Effervescent agent | | | | Content of | Fragrant material | |
|---|---|---|---|---|---|---|---|---|
| | Porous | | Sodium | | | magnesium | | Load amount |
| | micro- | Citric | hydrogen | Binding agent | | sulfate | | (g/Tablet |
| Ex. | particles | acid | carbonate | Type | Content | (anhydride) | Type | 1 g, Max) |
| Comp. | 2 | 5 | 5 | MCC | 10 | 10 | Lavender | 0.30 |
| Ex. 8 | | | | | | | | |
| Ex. 6 | 2 | 5 | 5 | MCC | 10 | 0 | Lavender | 0.45 |
| Ex. 10 | 2 | 5 | 5 | MCC | 10 | 1 | Lavender | 0.44 |
| Ex. 11 | 2 | 5 | 5 | MCC | 10 | 5 | Lavender | 0.45 |

TABLE 8

| | | | Post- | Post- |
|---|---|---|---|---|
| | | | effervescent | effervescent |
| | | | scent intensity | scent intensity |
| | Molding | Effervescent | after | after storage in |
| Ex | stability | property | manufacture | high humidity |
| Comp. Ex. 8 | ◯ | ◯ | 3 | 3 |
| Ex. 6 | ⊚ | ◯ | 4 | 3 |
| Ex. 10 | ◯ | ⊚ | 5 | 5 |
| Ex. 11 | ◯ | ⊚ | 5 | 5 |

As shown in Table 8, molding stability, effervescent property, and post-effervescent scent strength of the water-soluble effervescent solid perfume of Example 6 were good, and the water-soluble effervescent solid perfume according to Example 10 showed relatively superior results in all items compared to the sample according to Example 6. It can be determined that the reason that the effervescent property and the post-effervescent scent intensity after manufacture were excellent is that heat was generated when a water-soluble solid perfume which further comprises magnesium sulfate was added to water, thereby enhancing the effervescent properties, and consequently improving the immediate diffusion of scent. However, it was confirmed that if too much magnesium sulfate is added as in Comparative Example 8, the load rate of fragrance decreases and the content of the effervescent agent is relatively reduced, and thus the effervescent properties decrease and consequently the post-effervescent scent intensity decreases.

In summarizing the above results, the present invention not only can improve the load rate of the fragrant material and the scent stability, but also simultaneously improve the immediate diffusion of scent and the molding stability due to effervescent properties, while containing a high content of the fragrant material. Thereby, it is possible to provide various types of products that can satisfy the needs of consumers while maximizing the purpose of the product.

The above description is merely illustrative of the technical idea of the present invention, and those of ordinary skill in the art to which the present invention pertains can make various modifications and variations without departing from the essential characteristics of the present invention. In addition, the embodiments disclosed in the present invention are not intended to limit the technical idea of the present invention, but to explain the technical idea, and the scope of the technical idea of the present invention is not limited by these embodiments. The scope of protection of the present invention should be interpreted by the following claims, and all technical ideas within the scope equivalent thereto should be construed as being included in the scope of the present invention.

1. A water-soluble effervescent solid perfume comprising:
porous particles having a plurality of pores;
a fragrant material contained in the pores of the porous particles;
a binding agent for binding the porous particles to each other; and
an effervescent agent for dispersing the fragrant material in an aqueous system,
wherein a weight ratio of the porous particles to the binding agent is 1:1 to 30.

2. The water-soluble effervescent solid perfume according to claim 1, wherein the effervescent agent comprises an organic acid or an inorganic acid; and a carbonate salt, and wherein a weight ratio of the organic acid or inorganic acid:the carbonate salt is 1:
0.2 to 8.

3. The water-soluble effervescent solid perfume according to claim 1, wherein the solid perfume comprises:
based on the total weight of the porous particles and the binding agent and the effervescent agent,
1 to 30% by weight of the porous particles;
25 to 65% by weight of the binding agent; and
25 to 65% by weight of the effervescent agent.

4. The water-soluble effervescent solid perfume according to claim 1, wherein the porous particle is at least one selected from the group consisting of silica, zeolite, activated carbon and acrylic resin particles.

5. The water-soluble effervescent solid perfume according to claim 1, wherein the binding agent comprises at least one selected from the group consisting of hydroxypropyl methyl cellulose, microcrystalline cellulose, starch and polyvinyl pyrrolidone.

6. The water-soluble effervescent solid perfume according to claim 1, wherein an average particle size of the porous particle is 1 to 100 μm.

7. The water-soluble effervescent solid perfume according to claim 1, wherein an average pore diameter of the porous particle is 1 to 100 nm.

8. The water-soluble effervescent solid perfume according to claim 1, wherein the load rate of the fragrant material is 30 to 500% as calculated according to Equation 1:
Load rate (%)=(a maximum weight of loaded fragrant material (g)/a total weight of solid (g))×100, [Equation 1]
wherein the total weight of the solid represents the total sum of the weight of the porous particles, the weight of the binding agent and the weight of the effervescent agent.

9. The water-soluble effervescent solid perfume according to claim 1, wherein the structure formed by the porous particles and the binding agent has voids and the void has an average diameter of 0.1 to 100 μm.

10. The water-soluble effervescent solid perfume according to claim 1, wherein at least one selected from the group consisting of a cleaning agent, a sterilizer, and a deodorant is further contained in the pores of the porous particles.

11. The water-soluble effervescent solid perfume according to claim 1, wherein the perfume further comprises magnesium sulfate.

12. A method for manufacturing a water-soluble effervescent solid perfume, comprising mixing 3 to 100 parts by weight of porous particles, 30 to 300 parts by weight of a fragrant material, and 30 to 300 parts by weight of an effervescent agent based on 100 parts by weight of the binding agent to produce a mixture.

13. The method for manufacturing a water-soluble effervescent solid perfume according to claim 12, wherein the method further comprises preparing the mixture into the form of a tablet or capsule.

14. The method for manufacturing a water-soluble effervescent solid perfume according to claim 12, wherein the method further comprises mixing 1 to 30% by weight of magnesium sulfate based on the total weight of the porous particles and the binding agent and the effervescent agent.
